(19) United States
(12) Patent Application Publication
(10) Pub. No.: US 2016/0002661 A1
(43) Pub. Date: Jan. 7, 2016

(54) METHODS FOR IMPROVING DROUGHT TOLERANCE IN PLANTS

(71) Applicant: THE PENN STATE RESEARCH FOUNDATION, University Park, PA (US)

(72) Inventor: Jonathan P. Lynch, Boalsburg, PA (US)

(21) Appl. No.: 14/768,339

(22) PCT Filed: Mar. 3, 2014
(86) PCT No.: PCT/US2014/019955

§ 371 Date: Aug. 17, 2015

Related U.S. Application Data
(60) Provisional application No. 61/771,411, filed on Mar. 1, 2013.
(60) Provisional application No. 61/872,057, filed on Aug. 30, 2013.

(51) Int. Cl. C12N 15/82; A01H 1/02

(52) U.S. Cl. CPC C12N 15/8273; A01H 1/02

(57) ABSTRACT

Methods and materials for improving drought tolerance in plants are provided by identifying, selecting, and/or creating plants having reduced cortical cell file number (CCFN) or a larger average cortical cell size (CCS) area. Plants with a reduced CCFN or larger CCS area can be grown under low water conditions and have better plant growth and yield than corresponding plants with a higher CCFN or smaller CCS area.

### CROSS-REFERENCE TO RELATED APPLICATIONS

This application claims priority to U.S. Ser. No. 61/771,411, filed Mar. 1, 2013, and U.S. Ser. No. 61/872,057, filed Aug. 30, 2013, the disclosures of which are incorporated by reference in their entirety.

### STATEMENT REGARDING FEDERALLY SPONSORED RESEARCH

This invention was made with government support under Grant No. 0965380 awarded by the National Science Foundation, under Contract No. EDH-A-00-07000-05 awarded by the U.S. Agency for International Development (USAID), under Contract No. 2007-35100-18365 awarded by the United States Department of Agriculture/CSREES and under Hatch Act Project No. PEN04372, awarded by the United States Department of Agriculture/NIFA. The United States Government has certain rights in the invention.

### TECHNICAL FIELD

This invention relates to identifying, selecting, and/or creating plants with improved drought tolerance, and more particularly to identifying, selecting, and/or creating plants with reduced cortical cell file number (CCFN) or larger average cortical cell size (CCS) area. Plants with a reduced CCFN or larger CCS area can be grown under low water conditions and have better plant growth and yield than corresponding plants with a higher CCFN or smaller CCS.

### BACKGROUND

Maize production is facing major challenges as a result of the increasing frequency and intensity of drought events in several key production areas around the world (Tuberosa and Salvi, 2006, Trends Plant Sci 11: 405-412) and this problem will likely be exacerbated by climate change (Lobell et al., 2008, Science 333: 616-620). In developing countries, where the crop is mainly grown under rain-fed conditions, the problem of yield loss due to drought is most severe. Irrigation water availability in developed countries will decrease in the coming years due to climate change. Therefore, development of drought tolerant cultivars is an international issue of a strategic importance. This effort requires the identification and better understanding of specific phenes which improve crop drought tolerance.

### SUMMARY

This document is based on methods and materials for identifying, selecting, and/or creating plants with improved drought tolerance. For example, the methods and materials described herein can be used for identifying, selecting, and/or creating plants with reduced cortical cell file number (CCFN) or a large cortical cell size area (CCS). Plants with a reduced CCFN or large CCS can be grown under low water conditions and have better plant growth and yield than corresponding plants with a higher CCFN or smaller CCS. As described herein, there is substantial variation for CCS and CCFN in plants such as maize and this variation has a profound effect on root metabolic cost of soil exploration under drought. For example, a lower CCFN may reduce root respiration, thereby permitting greater root growth, water acquisition, plant growth and therefore drought tolerance. Accordingly, traits that can reduce metabolic costs are an important component of crop productivity under drought.

In one aspect, this document features a method for producing a drought tolerant plant. The method includes selecting a plant having (i) a reduced CCFN from a plurality of plants or (ii) a larger average CCS area from a plurality of plants; and producing a progeny of the selected plant. The progeny can be a seed, wherein upon planting the seed, the resulting plant is drought tolerant. The progeny can be produced by cross-pollinating the selected plant with a different plant of the same species. The progeny can be produced by self-pollinating the selected plant.

The plant can be a monocot. For example, the monocot can be selected from the group consisting of an Agrostis sps. (bent grass), an Andropogon sps. (blue stem grass), an Arundo sps. (cane), an Avena sps. (oats), a Cynodon sps. (Bermuda grass), an Elaeis sps. (oil palm), an Eragrostis sps. (love grass), a Festuca sps. (fescue), a Hordeum sps., a Lolium sps. (rye grass), a Miscanthus sps., an Oryza sps., a Panicum sps., a Pennisetum sps. (fountain grass), a Poa sps., a Saccharum sps., a Secale sps., a Sorghum sps., a Triticum sps. (wheat), a Zea sps., and a Zoysia sps. The plant can be a Hordeum vulgare, Oryza sativa, Panicum miliaceum, Panicum virgatum, Saccharum officinarum, Secale cereal, Sorghum bicolor, Triticum aestivum, Triticum durum, Triticum spelta, or Zea mays plant. For a Zea mays plant, the CCFN of the selected plant can be 10 or less (e.g. 9 or less or 6-8) and/or the CCS area of the selected plant can be greater than 230 μm² (e.g., greater than 300 μm², 350 μm², or greater than 400 μm²).

The plant can be a dicot. For example, the dicot can be selected from the group consisting of a Phaseolus sps., a Vigna sps., a Gossypium sps., a Medicago sps., a Helianthus sps., a Brassica sps., a Glycine sps., or a Carthamus sps. For example, the plant can be a Phaseolus vulgaris, Vigna radiata, Medicago sativa, Helianthus annuus, Brassica rapa, Brassica napus, Glycine max, or Carthamus tinctorius plant.

In another aspect, this document features a method of producing a maize plant. The method includes obtaining one or more first maize parent plants having (i) a root CCFN of 10 or less (e.g. 9 or less or 6-8) or (ii) an average root CCS area greater than 230 μm² (e.g., greater than 300 μm², 350 μm², or greater than 400 μm²); obtaining one or more second maize parent plants; and crossing the one or more first parent plants and the one or more second parent plants to produce progeny, wherein the progeny have drought tolerance. The first and/or second parent plants can be inbred lines.

This document also features a method of producing a maize plant. The method includes obtaining one or more first maize parent plants having (i) a root CCFN of 10 or less or (ii) an average root CCS area greater than 230 μm²; obtaining one or more second maize parent plants; crossing the one or more first parent plants and the one or more second parent plants; and selecting, for one to five generations, for progeny plants having drought tolerance.

Additionally, this document also features a seed that can produce a plant having a reduced CCFN or large CCS area. Plants selected as described herein can be used to produce such seeds.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and the drawings, and from the claims. The word “comprising” in the claims may be replaced by “consisting essentially of” or with “consisting of,” according to standard practice in patent law.

### DESCRIPTION OF DRAWINGS

FIG. 1 is a line graph of the change in soil moisture content at different depths (0.15 m, 0.30 m, 0.5 m) in well watered (WW) and water stressed (WS) plots. Terminal drought was imposed in WS plots beginning at 30 days after planting (DAP).

FIG. 2. is a histogram showing genetic variation for root cortical cell size of 78 Malawi maize landraces. The data shown are from standard reference tissue collected from 10-20 cm from the base of the second nodal crown root at 70 days after planting. Superimposed is the density plot for the normal distribution and using a kernel density estimate.

FIG. 3 is a graph of the correlation of root respiration per unit length and cortical cell size for GH1 (r2=0.46, p=0.009), GH2 (r2=0.59, p=0.001) and in GH3 (r2=0.52, p=0.018) in the mesocosms 30 days after planting. Each point is the mean of at least three measurements of respiration.

FIG. 4 is a graph of the correlation of root depth (D₉₅) and cortical cell size for GH2 (r²=0.48, p=0.001) and GH3 (r²=0.45, p=0.01) in the mesocosms 30 days after planting. The regression line is only shown for the significant relationships. Data include both water stressed (WS) and well watered (WW) conditions. D₉₅ measures the depth above where 95% of root length is present in mesocosms.

FIG. 5 is a bar graph of the stomatal conductance of lines with large and small CCS at 30 days after planting in the mesocosms. Data shown are means±SE of the means. Means with the same letters are not significantly different (p<0.05).

FIG. 6 is a graph of the relationship of rooting depth (D95) and stomatal conductance at 30 days after planting in mesocosms. Data include both water stressed (WS, open circles) and well watered (WW, closed circles). The regression line is only shown for the significant relationship.

FIGS. 7A and 7B are graphs of the relationship of cortical cell size and rooting depth (D95) in the field during two consecutive summers in Pennsylvania designated PA1 and PA2 (A and B, respectively). Data include both in water stress (WS, open circles) and well watered (WW, solid circles) conditions. D95 is the depth above which 95% of the roots were located in the soil profile. The regression line is only shown for the significant relationship.

FIGS. 8A, 8B, and 8C are graphs of the leaf relative water content at 60 days after planting (DAP) in the field during two consecutive summers in Pennsylvania, designated PA1 and PA2 (A and B, respectively) or in Malawi (C), both in well-watered (WW) and water-stressed (WS) conditions. Data shown are means±SE of the mean. Means with the same letters are not significantly different (P<0.05)

FIG. 9 is a bar graph of the shoot biomass of large and small cortical cells lines at 30 days after planting in EXP 1 and EXP2 (A and B, respectively) in the mesocosms. Data shown are means±SE of the means. Means with the same letters are not significantly different (p<0.05)

FIGS. 10A, 10B, and 10C are graphs of the shoot biomass in the field 70 days after planting in the field during two consecutive summers in Pennsylvania designated PA1 and PA2 (A and B, respectively), or in Malawi (C), both in water stress (WS) and well watered (WW) conditions. Data shown are means±SE of the means. Means with the same letters are not significantly different (p<0.05).

FIGS. 11A and 11B are graphs of grain yield in the field during two consecutive summers in Pennsylvania designated PA1 and PA2 (A and B, respectively), or in Malawi (C), both in water stress (WS) and well watered (WW) conditions. Data shown are means±SE of the means. Means with the same letters are not significantly different (p<0.05).

FIGS. 12A and 12B are graphs of the phenotypic variation for cortical cell file number (CCFN) in maize. FIG. 12A depicts 79 local landraces collected across Malawi. FIG. 12B depicts 70 recombinant inbred lines from Malawi maize breeding program grown in the field. Samples were collected at 70-80 days after planting.

FIG. 12C contains representative cross sections of maize roots showing genotypic difference in CCFN. Sections are from maize crown roots grown in the field 70 days after planting. Images were obtained from laser ablation tomography.

FIG. 13 is a graph of the relationship of CCFN and root segment respiration at 30 days after planting in the mesocosms. Data include 14 IBM lines (closed circles) and 16 NyH lines (open circles). The fitting line is only included in the significant relationships: r2 and p-value are shown; *, p<0.05

FIG. 14 is a graph of the relationship of CCFN and rooting depth (D₉₅) at 30 days after planting in the mesocosms in experiment II. Data include both water stressed (WS, closed circles) and well watered (WW, open circles). D₉₅ measures the depth where 95% of root length in mesocosms. The fitting line is only included in the significant relationships: r2 and p-value are shown; ***, p<0.001.

FIG. 15 is a bar graph of the stomatal conductance of six lines with contrasting CCFN at 30 days after planting in the mesocosms. Data shown are means±SE of the means. Means with the same letters are not significantly different (p<0.05).

FIGS. 16A and 16B are bar graphs of the shoot dry weight at 30 days after planting of six IBM lines in well watered (WW) and water stressed (WS) conditions in mesocosms during experiment II (A) and experiment III (B). Bars are means±SE of the mean (n=4). Means with the same letters are not significantly different within the same panel (p<0.05).

FIG. 17 is a graph of the relationship of CCFN and rooting depth (D₉₅) at 70 days after planting in the mesocosms in field 1 experiment in Rock Springs. Data include both water stressed (WS, closed circles) and well watered (WW, open circles). D₉₅ measures the depth where 95% of root length in soil profile. The fitting line is only included in the significant relationships: r2 and p-value are shown; ***, p<0.001.

FIGS. 18A-18F are bar graphs of the performance of maize lines contrasting in CCFN in water stress (WS) and well watered (WW) conditions in the rainout shelters at Rock Springs, Pa., USA. FIGS. 18A and 18B are the leaf relative water content at 60 days after planting in experiments in field 1 and field 2 experiments, respectively. FIGS. 18C and 18D are the shoot biomass per plant at 70 days after planting in field 1 and field 2 experiments, respectively. FIGS. 18E and 18F are the in field 1 and field 2 experiments, respectively. Bars show means±SE of four replicates per treatment. Means with the same letters are not significantly different within the same panel (p<0.05).

FIGS. 19A-19F are bar graphs of the performance of maize lines contrasting in CCFN in the field in water stress (WS) and well watered (WW) conditions at in two agroecologies in Malawi; Bunda (A,C,E) and Chitala (B,D,F). FIGS. 19A and 19B depict leaf relative water content at 60 days after planting; FIGS. 19C and 19D depict shoot biomass per plant at 70 days after planting; and FIGS. 19E and 19F depict yield per plant. Bars show means±SE (n=16-18) of four replicates per treatment and trait. Means with the same letters are not significantly different within the same panel (p<0.05).

### DETAILED DESCRIPTION

This document is based on methods and materials for identifying, selecting, and/or creating plants with improved drought tolerance. For example, the methods and materials described herein can be used for identifying, selecting, and/or creating plants with a reduced cortical cell file number (CCFN) or large average root cortical cell size area (CCS). Plants with a reduced CCFN or larger CCS area can be grown under low water conditions and have better plant growth and yield than corresponding plants with a higher CCFN or smaller CCS. As described herein, there is substantial variation for CCS and CCFN in plants such as maize and this variation has a profound effect on root metabolic cost of soil exploration under drought, both in terms of the carbon cost of root respiration as well as the nutrient content of living tissue. For example, a lower CCFN may reduce root respiration, thereby permitting greater root growth, water acquisition, plant growth and therefore drought tolerance. As CCFN can be easily observed with a microscope, it is amenable to direct phenotypic selection in crop improvement programs. Larger CCS may improve drought tolerance by reducing root metabolic cost, permitting greater root growth and water acquisition from drying and ultimately improving plant growth (e.g., biomass) and yield (e.g. grain yield). Accordingly, traits that can reduce metabolic costs are an important component of crop productivity under drought.

Methods described herein include selecting a plant having a reduced root CCFN or a large average root CCS area from a plurality of plants (e.g., two or more plants). CCS area can be determined, for example, by examining a cross-section of tissue of the root cortex using, for example, laser ablation tomography and estimating the cell size in the center of the cortex (e.g., mid-cortical band). As described herein for maize, the CCS area can range from about 100 μm² to about 551 μm², with plants having a small CCS area ranging from 127 to 217 μm² (mean=166±5) and plants having a large CCS area ranging from 239 to 551 μm² (mean=411±16). Thus, for maize, a plant can be identified as having a large CCC when the average CCS area is about 230 μm² or greater. For example, a plant can be identified as having an average CCS area of 300 μm², 350 μm² 400 μm², 450 μm², or 500 μm² or more and used in the methods described herein. One of ordinary skill in the art will appreciate that the CCS area for a species other than maize may differ, but the CCS area is readily determinable using the methodology described herein.

CCFN also can be determined using a microscope or laser ablation tomography and counting the cell layers from the epidermis to the endodermis. As described herein for maize, the CCFN ranged from 6 to 19, with plants identified as having a reduced CCFN when the CCFN was 10 or less (e.g., 9 or less, 8 or less, 7 or less, such as a CCFN of 6 to 8). One of ordinary skill in the art will appreciate that the CCFN for a species other than maize may differ, but CCFN is readily determinable using the methodology described herein.

Once a plant having a reduced CCFN or large CCS size is identified, the plant can be grown, or it can be bred to produce other plants or seeds having this phenotype. The plant can be bred sexually or asexually by means known within the art. Examples of sexual means to breed a plant include self-pollination and cross-pollination. Examples of asexual means to reproduce the plant include budding, tillering, and apomixis. One of ordinary skill within the art would appreciate that self-pollinated or asexually reproducing the plant having a reduced CCFN or larger CCS area will produce a copy of the plant. Therefore, there is a greater likelihood that the self-pollinated or asexually reproduced plant would have the improved drought tolerance.

Reproducing a plant, whether by sexual or asexual means, produces a progeny. The progeny can be in the form of a seed produced through the sexual or asexual reproductive process, or a plant produced through certain forms of asexual production, such as tillering. Through plant breeding, one of ordinary skill would be able to preserve the reduced CCFN or larger CCS area in the progeny and subsequent generations.

For example, the progeny can be a seed, wherein upon planting the seed, the resulting plant can be drought tolerant and have increased yield under low water conditions. For example, grain yield from plants with a larger CCS area can be increased at least 10%, at least 20%, at least 30%, at least 40%, or at least 50% relative to a corresponding plant with a smaller CCS area.

The plants described herein may be used in a plant breeding program. Any of a number of standard breeding techniques can be used for breeding according to the selection of a trait, i.e., reduced CCFN or larger CCS area, depending upon the species to be crossed. The goal of plant breeding is to combine, in a single variety or hybrid, various desirable traits, wherein the reduced CCFN or larger CCS area is at least one of the desired traits.

This document encompasses methods for identifying a plant having a reduced CCFN or large CCS area, reproducing that plant and/or producing a new plant by crossing a first parent plant with a second parent plant wherein one or both of the parent plants is a plant having a reduced CCFN or larger CCS. For example, a plant having a reduced CCFN or larger CCS can be identified in a first plant.

Plant breeding techniques known in the art and used in a plant breeding program include, but are not limited to, recurrent selection, bulk selection, mass selection, backcrossing, pedigree breeding, open pollination breeding, restriction fragment length polymorphism enhanced selection, genetic marker enhanced selection, doubled haploids, and transformation. Often, combinations of these techniques are used.

The development of hybrids in a plant breeding program requires, in general, the development of homozygous inbred lines, the crossing of these lines, and the evaluation of the crosses. There are many analytical methods available to evaluate the result of a cross. The oldest and most traditional method of analysis is the observation of phenotypic traits. Alternatively, the genotype of a plant can be examined.

A genetic trait which has been identified, selected or engineered into a particular plant using breeding or transformation techniques can be moved into another line using traditional breeding techniques that are well known in the plant breeding arts. For example, a backcrossing approach is commonly used to move a trait from a one maize plant to an elite inbred line, and the resulting progeny would then comprise the trait(s). Also, if an inbred line was used for trait selection, then the plants could be crossed to a different inbred line in order to produce a hybrid maize plant. As used herein, “crossing” can refer to a simple X by Y cross, or the process of backcrossing, depending on the context.

The development of a hybrid in a plant breeding program involves three steps: (1) the selection of plants from various germplasm pools for initial breeding crosses; (2) the self-crossing of the selected plants from the breeding crosses for several generations to produce a series of inbred lines, which, while different from each other, breed true and are highly homozygous; and (3) crossing the selected inbred lines with different inbred lines to produce the hybrids. During the inbreeding process, the vigor of the lines decreases. Vigor is restored when two different inbred lines are crossed to produce the hybrid. An important consequence of the homozygosity and homogeneity of the inbred lines is that the hybrid created by crossing a defined pair of inbreds will always be the same. Once the inbreds that give a superior hybrid have been identified, the hybrid seed can be reproduced indefinitely as long as the homogeneity of the inbred parents is maintained.

In some embodiments, the methods are directed to breeding a plant line. Such methods can use genetic polymorphisms identified as described herein in a marker assisted breeding program to facilitate the development of lines that have a desired alteration in drought tolerance. Once a suitable genetic polymorphism is identified as being associated with variation for the trait, one or more individual plants are identified that possess the polymorphic allele correlated with the desired variation. Those plants are then used in a breeding program to combine the polymorphic allele with a plurality of other alleles at other loci that are correlated with the desired variation. Techniques suitable for use in a plant breeding program are known in the art and include, without limitation, backcrossing, mass selection, pedigree breeding, bulk selection, crossing to another population and recurrent selection. These techniques can be used alone or in combination with one or more other techniques in a breeding program. Thus, each identified plants is selfed or crossed a different plant to produce seed which is then germinated to form progeny plants. At least one such progeny plant is then selfed or crossed with a different plant to form a subsequent progeny generation. The breeding program can repeat the steps of selfing or outcrossing for an additional 0 to 5 generations as appropriate in order to achieve the desired uniformity and stability in the resulting plant line, which retains the polymorphic allele. In most breeding programs, analysis for the particular polymorphic allele will be carried out in each generation, although analysis can be carried out in alternate generations if desired.

In some cases, selection for other useful traits is also carried out, e.g., selection for disease resistance. Selection for such other traits can be carried out before, during or after identification of individual plants that possess the desired polymorphic allele.

The above described method is applicable to any monocot or dicot plant. Non-limiting examples of monocots include Agrostis sps. (bent grass), Andropogon sps. (blue stem grass), Arundo sps. (cane), Avena sps. (oats), Cynodon sps. (Bermuda grass), Elaeis sps. (oil palm), Eragrostis sps. (love grass), Festuca sps. (fescue), Hordeum sps., Lolium sps. (rye grass), Miscanthus sps., Oryza sps., Panicum sps., Pennisetum sps. (millets), Poa sps., Saccharum sps., Secale sps., Sorghum sps., Triticum sps. (wheat), Zea sps., or a Zoysia sps. For example, the plant can be Hordeum vulgare (barley), Oryza sativa (rice), Panicum miliaceum, Panicum virgatum, Saccharum officinarum, Secale cereal, Sorghum bicolor, Pennisetum glaucum, Triticum aestivum, Triticum durum, Triticum spelta, or Zea mays (maize). The methods may be particularly useful for maize and other graminaceous crop species lacking secondary root growth, including rice (Oryza sativa), wheat (Triticum aestivum L.), barley (Hordeum vulgare L.), oats (Avena sativa), sorghum (Sorghum bicolor), and millet (Pennisetum glaucum).

Non-limiting examples of dicots include Phaseolus sps., Vigna sps., Gossypium sps., Medicago sps., Helianthus sps., Brassica sps., Glycine sps., or Carthamus sps. For example, the plant can be Phaseolus vulgaris (bean), Vigna radiate (Mung bean), Medicago sativa (alfalfa), Helianthus annuus (sunflower), Brassica rapa, Brassica napus (canola), Glycine max (soybean), or Carthamus tinctorius (safflower).

The invention is further described in the following examples, which do not limit the scope of the invention described in the claims.

### EXAMPLES

### Example 1

### Materials and Methods for Assessing Root Cortical Cell Size Under Drought Plant Materials

Based on preliminary experiments conducted under optimal conditions in the field and greenhouse, a set of six IBM lines contrasting in CCS was selected for experiments for one year and another set of six IBM lines also contrasting in CCS was selected for experiments in the consecutive year (Table 1). The IBM lines are from the intermated population of B73xMo17 and were obtained from the University of Wisconsin, Madison, Wis., USA (Genetics Cooperation Stock Center, Urbana, Ill., USA) and designated as Mo (Table 1); NyH are from the Ny821xH99 population (University of Wisconsin, Madison, Wis., USA). Another set of 10 lines was used to assess the impact of phenotypic variation of CCS on root respiration. In Malawi, the experimental material consisted of a set of 6 lines (Table 1). The small CCS selection group had cell sizes from 127 to 217 (mean=166±5) and large CCS selection group from 239 to 551 (mean=411±16).

TABLE 1
Summary of the experiments

| | | | | Selection | |
|---|---|---|---|---|---|
| | | | | group based | |
| Experiment | *ID | Environment | Rep | on CCS | Plant material |
| Experiment 1 | EXPI | Greenhouse | 3 | NA | |
| Experiment 2 | EXPII | Greenhouse | 4 | Large CCS | Mo178, 059, 026 |
| | | | | Small CCS | Mo284, 181 |
| Experiment 3 | EXPIII | Greenhouse | 4 | Large CCS | Mo201, 090, 126 |
| | | | | Small CCS | Mo039, 323, 344 |
| Experiment 4 | PA1 | Field-PA, USA | 4 | Large CCS | Mo178, 059, 026 |
| | | | | Small CCS | Mo284, 181 |
| Experiment 5 | PA2 | Field-PA, USA | 4 | Large CCS | Mo201, 090, 126 |
| | | | | Small CCS | Mo039, 323, 344 |
| Experiment 6 | MW2-1 | Field-Bunda, MW | | NA | |
| Experiment 7 | MW2-2 | Field-Bunda, MW | 4 | Large CCS | MW297, 386, 696 |
| | | | | Small CCS | MW1772, 218, 629 |

*based on year and where the experiment was conducted

Greenhouse Experiments

A total of three experiments were carried out under the same conditions in two consecutive years (Table 1). The experiments were conducted in a greenhouse at University Park, Pa., USA (77° 49′W, 40° 4′N) under constant conditions (14/10 h day/night: 23/20° C. day/night: 40-70% relative humidity), with maximum 1200 μmol photons m⁻² s⁻¹ PAR and additional light was provided when necessary with 400-W metal-halide bulbs (Energy Technics, York, Pa., USA). Plants were grown in mesocosms consisting of PVC cylinders 1.5 m in height by 0.154 m in diameter, with plastic liners made of 4-mil (0.116-mm) transparent hi-density polyethylene film, which was used to facilitate root sampling. The growth medium consisted of (by volume) 50% commercial grade sand (Quikrete Companies Inc. Harrisburg, Pa., USA), 35% vermiculite (Whittemore Companies Inc., Lawrence, Mass., USA), 5% Perlite (Whittemore Companies Inc., Harrisburg, Pa., USA), and 10% topsoil (Hagerstown silt loam top soil (fine, mixed, mesic Typic Hapludalf)). Mineral nutrients were provided by mixing the media with 70 g of OSMOCOTE PLUS fertilizer consisting of (in %); N (15), P (9), K (12), S (2.3), B (0.02) Cu (0.05), Fe (0.68), Mn (0.06), Mo (0.02), and Zn (0.05) (Scotts-Sierra Horticultural Products Company, Marysville, Ohio, USA) for each column. The seeds were germinated by placing them in darkness at 28±1° C. in a germination chamber for two days prior to transplanting two seedlings per mesocosm, thinned to one per mesocosm 5 days after planting.

At harvest (i.e., 30 days after planting), the shoot was removed, and the plastic liner was pulled out of the PVC column and laid on a washing bench. The plastic liner was cut open and the roots were washed carefully by rinsing the media away with water. This allowed us to recover the entire plant root system. Samples for root respiration measurement were collected from 10-20 cm from the base of three representative second whorl crown roots per plant. Root respiration (CO₂ production) was measured using Li-Cor 6400 (Li-Cor Biosciences, Lincoln, Nebr., USA) equipped with a 56 ml chamber. The change in CO₂ concentration in the chamber was monitored for 3 minutes. During the time of measurement the chamber was placed in a temperature controlled water bath at 27±1° C. to control temperature fluctuations. Following respiration measurements, root segments were preserved in 75% ethanol for anatomical analysis as described below.

Root length distribution was measured by cutting the root system into 7 segments of 20 cm depth increments. Roots from each increment were spread in a 5 mm layer of water in transparent plexiglass trays and imaged with a flatbed scanner equipped with top lighting (Epson Perfection V700 Photo, Epson America, Inc. USA) at a resolution of 23.6 pixel mm⁻¹ (600 dpi). Total root length for each segment was quantified using WinRhizo Pro (Regent Instruments, Québec City, Québec, Canada). Following scanning the roots were dried at 70° C. for 72 hours and weighed. To summarize the vertical distribution of the root length density we used the D95 (Schenk et al., 2002), i.e. the depth above which 95% of the roots were located in the column.

Root segments that were used for respiration measurements were ablated using laser ablation tomography, which is a semi-automated system that uses a laser beam to vaporize or sublimate the root at the camera focal plane ahead of an imaging stage. The sample is incremented, vaporized or sublimated, and imaged simultaneously. The cross-section images were taken using a Canon T3i (Canon Inc. Tokyo, Japan) camera with 5× micro lens (MP-E 65 mm) on the laser-illuminated surface. Root images were analyzed using RootScan, an image analysis tool developed for analyzing root anatomy (Burton et al., 2012). The CCS was determined from three different images per root segment. CCS was calculated as a median cell size.

### Experiment I (EXP1)

A randomized complete block design (RCBD) was used in this experiment, with time of planting as a blocking factor replicated three times. A set of 10 IBM lines (Table 1) was planted under mild water stress. Water stress was imposed by withholding water 14 days after planting. Plants were harvested for root respiration measurements 35 days after planting.

### Experiment II (EXPII) and III (EXPIII)

Two experiments were conducted, one in the fall (EXPII) and one in the following summer (EXPIII). A set of six genotypes was planted in each experiment (Table 1). A randomized complete block design with time of planting as a blocking factor replicated four times was used in both experiments. Planting was staggered by 7 days. In both experiments, the irrigated mesocosms (control) each received 200 ml of water every other day, to replenish water lost by evapotranspiration, and in stressed mesocosms, water application was withheld 5 days after planting to allow the plants to exploit residual moisture to simulate terminal drought. An SC-1 leaf porometer (Decagon, Pullman, Wash.) was used for stomatal conductance measurements from the abaxial sides of third fully expanded 28 days after planting in EXPIII. All of the measurements were made between 0900 h and 1100 h. Plants were harvested 30 days after planting for root respiration measurements, root growth distribution and shoot biomass. The dry matter of the shoot and root were measured after drying at 70° C. for 72 h and root length distribution was determined as described above.

Field Experiments Rock Springs, Pa., USA

Field Sites and Experimental Setup

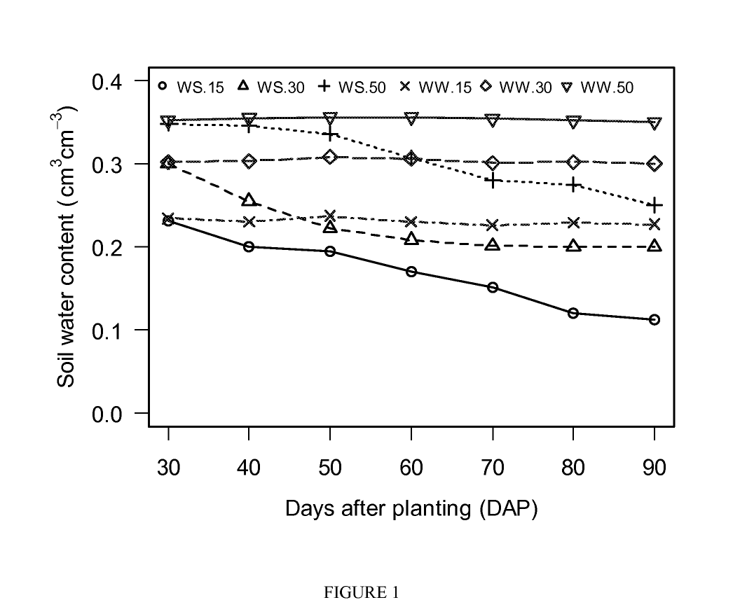

Two experiments were conducted in rainout shelters located at the Russell E. Larson Agricultural Research Center in Rock Springs, Pa., USA (77° 57′W, 40° 42′N,), during two consecutive summers (designated PA1 and PA2). The soil is a Hagerstown silt loam (fine, mixed, mesic Typic Hapludalf). Both experiments were arranged as split-plots in a randomized complete block design with four replications. The main plots were composed of two moisture regimes and the subplots contained six lines contrasting in cortical cell size in each experiment. Each subplot consisted of three rows, with each row being 2.5 m long, with a row spacing of 0.75 m. The drought treatment was initiated 35-40 days after planting using an automated rainout shelter. The shelters (10 by 30 m) were covered with a clear greenhouse plastic film (0.184 mm) and were automatically triggered by rainfall to cover the plots, and excluding natural precipitation throughout the entire growing season. The shelters automatically opened quickly after rainfall, exposing experimental plots to natural ambient conditions whenever it was not raining Adjacent non-sheltered control plots were rainfed and drip-irrigated when necessary to maintain the soil moisture close to field capacity throughout the growing season. The depleting moisture content within root zone at different soil depths (20, 35 and 50 cm) was monitored at regular intervals (FIG. 1), using TRIME FM system (IMKO, GmbH, Ettlingen, Germany) both inside and outside the rainout shelter.

Plant Measurements

Leaf relative water content (RWC) was measured and used as a physiological indicator of plant water status. To measure leaf RWC, fresh leaf discs (3 cm in diameter) were collected from the third fully expanded leaf for three representative plants per plot 60 days after planting and weighed immediately to determine fresh weight (FW). The discs were then soaked in distilled water for 12 h at 4° C. with minimal light. Following soaking, the discs were blotted dry and again weighed to determine turgid weight (TW). After being dried in an oven at 70° C. for 72 h, discs were weighed again for dry weight (DW). Leaf RWC was calculated according to the Barrs and Weatherley method (Aust J Biol Sci 15: 413-428 (1962)).

Root growth and distribution was evaluated by collecting coil cores 80 days after planting. A soil coring tube (Giddings Machine Co., Windsor, Colo., USA) 5.1 cm in diameter and 60 cm long was used for sampling, the core was taken midway between the plants within a row. The cores were sectioned into 6 segments of 10 cm depth increments and washed. Subsequently the washed roots were scanned using a flatbed scanner (Epson, Perfection V700 Photo, Epson America, Inc. USA) at a resolution of 23.6 pixel mm⁻¹ (600 dpi) and analyzed using image processing software WinRhizo Pro (Regent Instruments, Québec city, Québec, Canada).

Shoot and roots were evaluated 75 days after planting. To accomplish this, three representative plants in each plot were cut at soil level. The collected shoot material was dried at 70° C. for 72 hours and weighed. Root crowns were excavated by the ‘shovelomics’ method (see Trachsel et al., Plant Soil, 341: 75-87 (2010)). Three 8-cm root segments were collected 10-20 cm from the base of a second whorl crown root of each plant, and used to assess cortical cell size. The segments were preserved in 75% ethanol before being processed as described above. At physiological maturity, grain yield was collected from 10 bordered plants per plot.

Field Experiments—Malawi

Assessing Phenotypic Variation of CCS in Malawi Germplasm (MW2-1)

A set of 81 maize landrace collected across Malawi were obtained from the Department of Agricultural Research and Technical Services, Malawi and planted at Bunda (33° 48′E, 14° 10'S) under optimum conditions (i.e., the plots were rainfed but only rarely were they severely moisture stressed). The experiment was arranged as randomized complete design with three replications. Each plot consisted of a single 6 m long row with 25 plants. Root crowns were excavated by ‘shovelomics’ (Trachsel et al., 2010, supra). Three 8-cm root segments were collected 10-20 cm from the base of a representative second whorl crown root of each plant, and used to assess CCS. The segments were preserved in 75% ethanol before being processed as described above.

Utility on CCS Under Water Limited Condition (MW2-2)

The field experiment was conducted at Bunda College research farm, Lilongwe, Malawi (33° 48′E, 14° 10'S) during summer (i.e., the rain-free period August to November). The soil is an Oxic Rhodustalfs. A set of 6 maize genotypes contrasting in CCS was planted (Table 1). The experiment was arranged as split-plot in a randomized complete block design with four replications. The main plots were composed of two moisture regimes and the subplots contained 6 genotypes contrasting in CCS. Seeds were planted in 6 m row plots with 25 cm and 75 cm spacing between planting stations and rows respectively. At planting, both the control and stressed plots received the recommended amounts of irrigation. Drought stress was managed by withholding irrigation six weeks after planting so that moisture stress was severe enough to reduce yield and shoot biomass by 30-70%. Control plots, which received supplementary irrigation, were planted alongside the stress plots separated by a 5 m wide alley. At each location, the recommended fertilizer rate was applied during planting and top dressed three weeks after planting. Leaf relative water content was determined 60 days after planting as described above. Shoot and roots were evaluated 75 days after planting. The collected shoot material was dried at 70° C. for 72 hours and weighed. Root crowns were excavated by ‘shovelomics’ (Trachsel et al., 2010, supra). Three 8-cm root segments were collected 10-20 cm from the base of a representative second whorl crown root of each plant, and used to assess CCS. The segments were preserved in 75% ethanol before being processed as described above. At physiological maturity, grain yield was collected each plot.

Data Analysis

The data from each year were analyzed separately since different sets of genotypes were used. For greenhouse data, for comparisons of genotypes, irrigation levels and their interaction effects, a two-way analysis of variance (ANOVA) was used. Field data were analyzed as randomized complete block split plot design to determine the presence of significant effects due to soil moisture regime, genotype (or selection group) and interaction effects on the measured and calculated parameters. Mean separation of genotypes for the different parameters was performed by a Tukey-HSD test. Unless otherwise noted, HSD_{0.05} values were only reported when the F-test was significant at P≦0.05. Linear regression analysis was used to establish relationships between CCS and measured or calculated parameters. Data was analyzed using R version 3.0.0 (R Development Core Team).

### Example 2

### Phenotypic Variation of CCS and Root Respiration

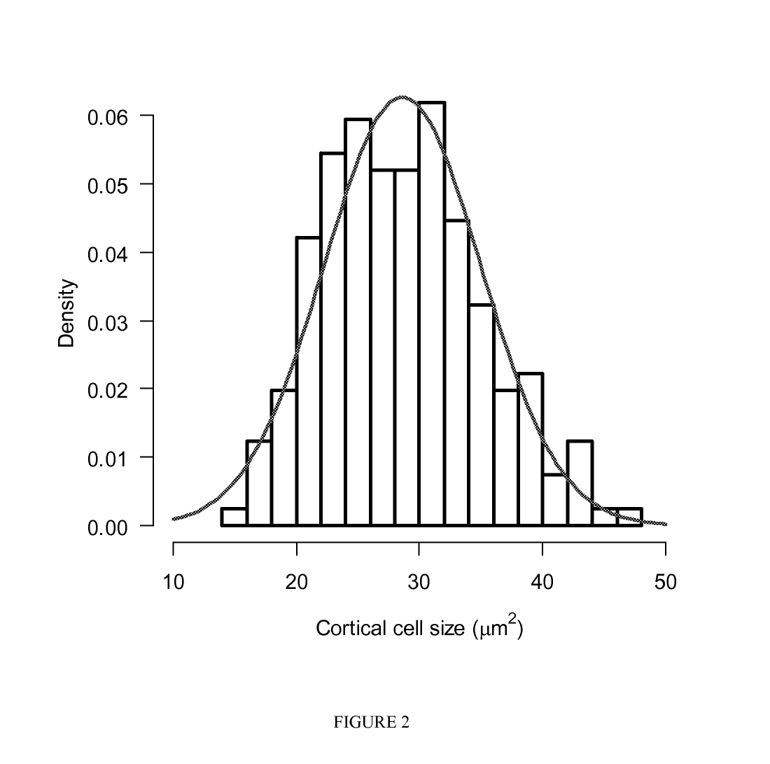

The maize root cortex is comprised of homogeneous parenchyma type cells. There is a variation of cell sizes across the root cortex with cells close to the epidermis being small, increasing in size towards the middle of the cortex. Towards the inner cortex close to the endodermis, the cortex cells become smaller. In this study, the median cell size for the mid-cortical region was chosen as a representative value for the root CCS. It was observed that there was considerable phenotypic variation for CCS Malawian landraces. The CCS variation is over 300% in maize, with the largest cells 500 μm² and smallest cells 150 μm²′ based on a standard reference tissue collected from 10-20 cm from the base of the second nodal crown root (FIG. 2).

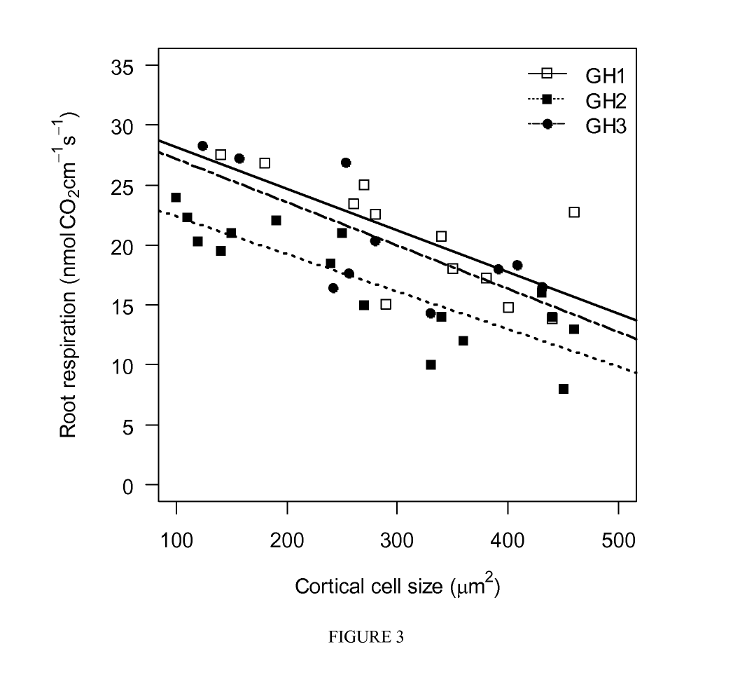

The respiration rate of root segments was measured in greenhouse experiment I (EXPI), II (EXPII) and III (EXPIII). From the combined results, there was a strong negative correlation between root cortical cell size and respiration (FIG. 3). On average, lines with large cells had 59% less root respiration than lines with small cells. CCS explained 53% of the observed variation in respiration rate (FIG. 3).

Effect of CCS on Root Growth and Plant Water Status

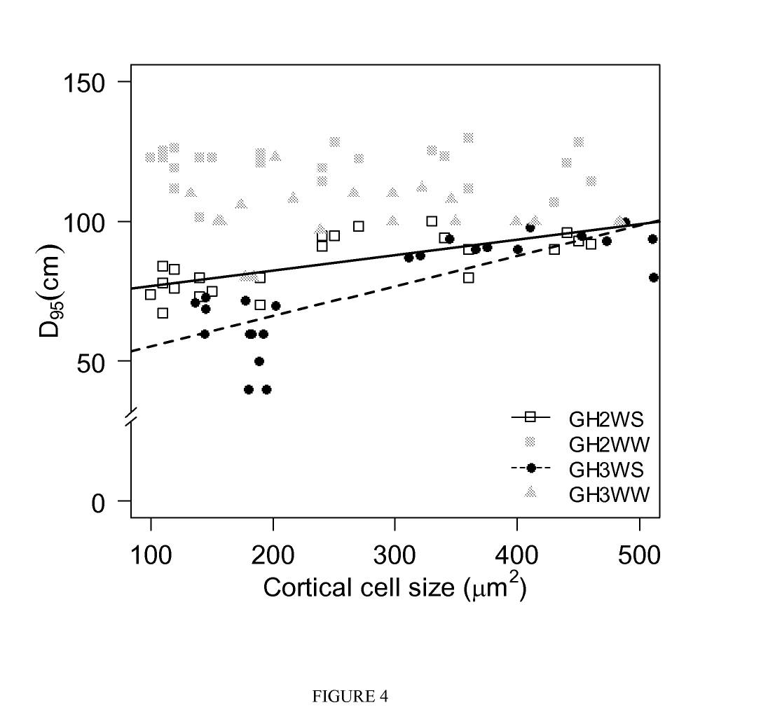

In the greenhouse, water stress significantly reduced rooting depth (D₉₅) 30 days after planting in EXPII and EXPIII (Table 2). CCS was positively correlated to rooting depth (D₉₅) under water stress and there was no relationship in well-watered conditions (FIG. 4). Under water stress, lines with large CCS had more roots deeper in the columns. On average the D₉₅ for lines with large CCS was 21% deeper in EXPII and 27% deeper in EXPIII than lines with small CCS.

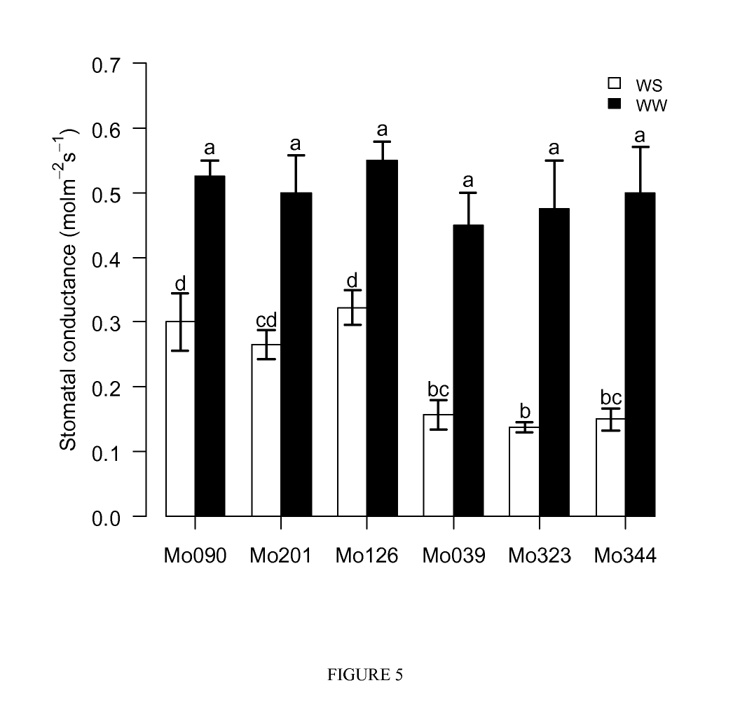

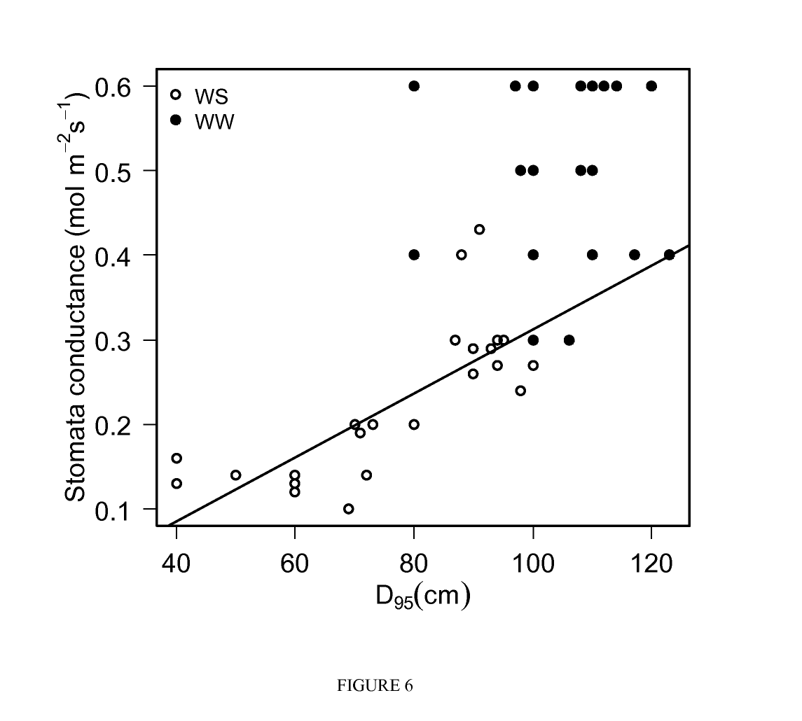

In EXPIII, stomatal conductance was significantly reduced by water stress, a 68% reduction relative to well-watered plants 30 days after planting in the mesocosms (Table 1; FIG. 5). Under water stress, lines with large CCS had 50% greater stomata conductance than lines with small CCS (FIG. 5). Linear regression was used to estimate the effect of rooting depth on plant water status in the mesocosms. Stomata conductance was positively correlated with D₉₅ in water-stressed conditions and there was no relationship in well-watered conditions (FIG. 6).

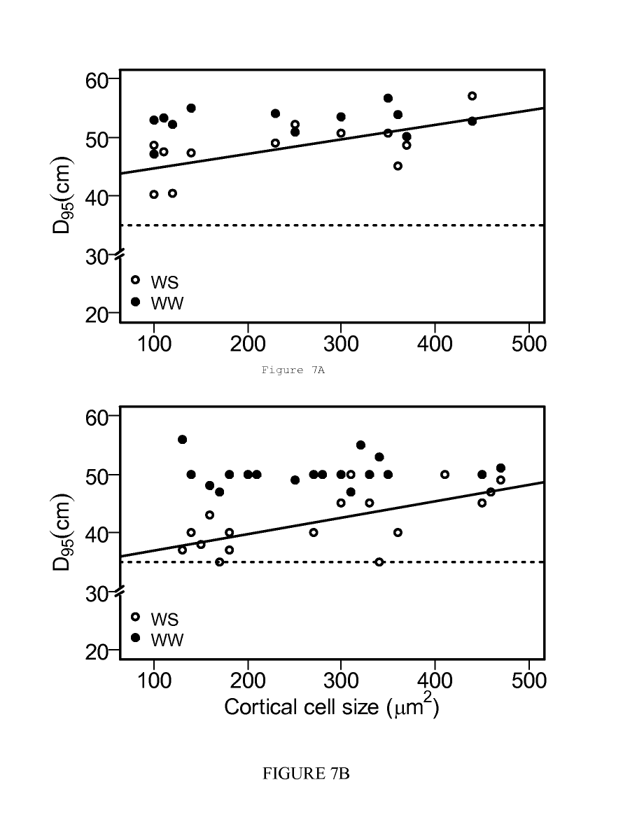

In the field under water stress CCS was positively correlated with rooting depth (D₉₅) in both experiments (FIGS. 7A and 7B). However, there was no relationship between CCS and rooting depth in well-watered conditions (FIG. 7). Under water stress in USA (PA1) lines with large cells had 41% greater rooting depth than lines with small CCS while in the next year (PA2) lines with large CCS had 32% deeper D₉₅ than lines with small CCS.

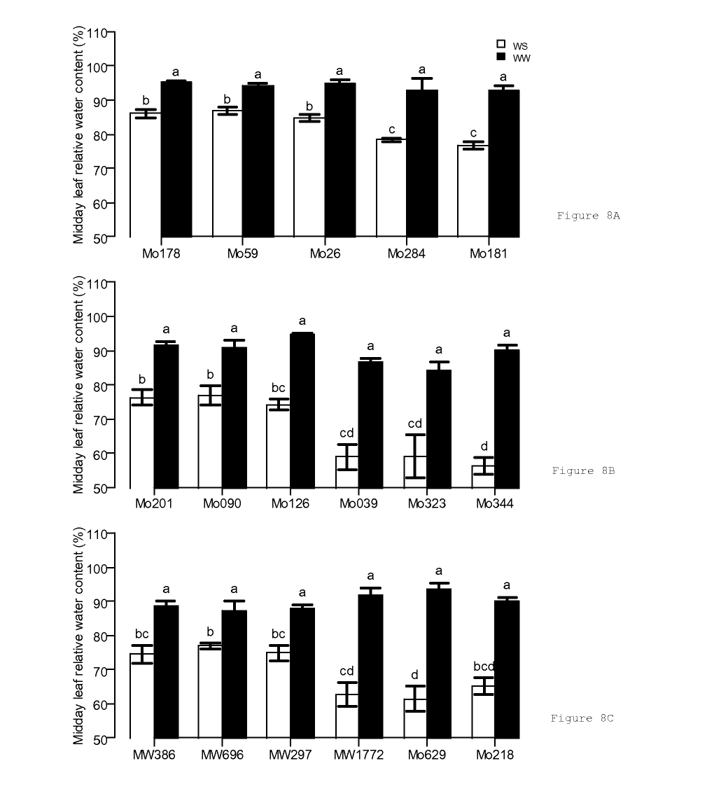

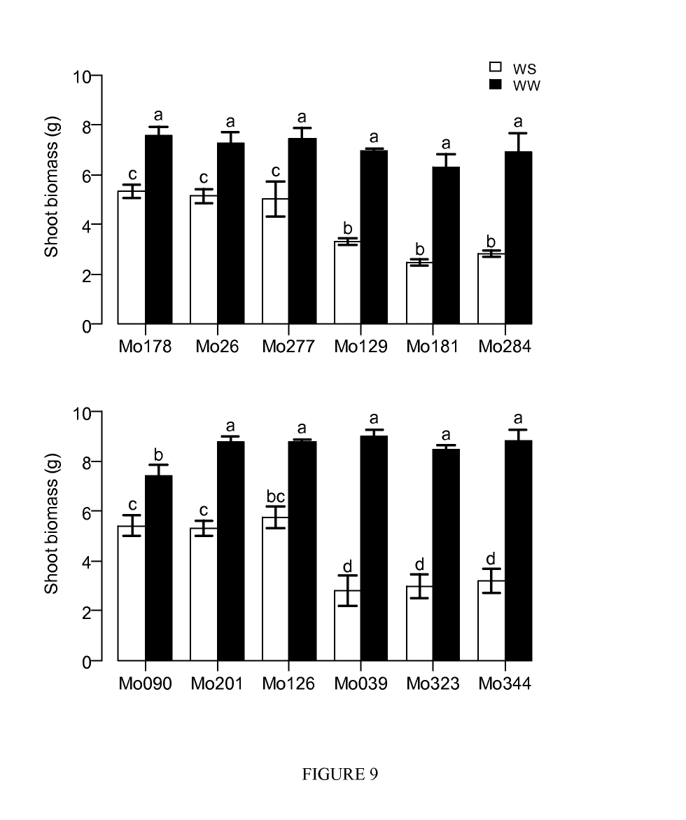

Midday leaf relative water content of well-watered plants 60 days after planting averaged approximately 93%, with no differences among genotypes (FIG. 8A-8C). Water stress significantly reduced leaf relative water content in all field experiments (Table 3 and FIG. 8). Under water stress, lines with large CCS had greater leaf relative water content than line with small CCS by 22% (EXP1), 30% (EXP2) and 20% (MW2-2) (FIGS. 9A and 9B).

TABLE 2
Summary (F and P values) of analysis of variance for the effects of water treatment and
genotype on shoot biomass, rooting depth (D95), and stomata conductance in mesocosms

| | Experiment II | | | Experiment III | | | |
|---|---|---|---|---|---|---|---|
| | | F ratio | F ratio | | F ratio | F ratio | F ratio |
| Source | df | Biomass | D95 | df | Biomass | D95 | Conductance |
| Irrigation | 1 | 51.04*** | 46.89*** | 1 | 32.47*** | 29.80*** | 16.02*** |
| RIL | 5 | 9.77*** | 6.91*** | 5 | 15.39*** | 15.29** | 5.09** |
| Irrigation * RIL | 5 | 2.70* | 6.75*** | 5 | 28.86*** | 11.86** | 0.72 |

**P from 0.05 to 0.01:
***P from 0.01 to 0.001.
D95 is the root depth measures the depth where 95% of root length in mesocosms,
Irrigation is the moisture regimes imposed;
Conductance is the stomatal conductance (mol m⁻²s⁻¹)

TABLE 3
Summary (F and P values) of analysis of variance for the effects of water treatment and genotype on yield, shoot biomass, rooting
depth (D95), and leaf relative water content in field

| | PA1 | | | | PA2 | | | | | MW2-2 | | | |
|---|---|---|---|---|---|---|---|---|---|---|---|---|---|
| | | F ratio | | | | F ratio | | | | | F ratio | | |
| Source | df | Biomass | D₉₅ | RWC | df | Biomass | D₉₅ | RWC | Yield | df | RWC | Biomass | Yield |
| Irrigation | 1 | 49.5*** | 6.5* | 21.0*** | 1 | 41.3*** | 4.9* | 35.7* | 46.2*** | 1 | 58.8*** | 166.0*** | 81.49*** |
| Gen | 4 | 6.7*** | 7.2*** | 9.7*** | 5 | 3.9** | 0.6 | 9.8** | 10.1*** | 5 | 2.0* | 23.6*** | 20.0*** |
| Irrigation * Gen | 4 | 4.3** | 1.6*** | 5.7** | 5 | 5.5*** | 0.3*** | 4.2** | 10.7*** | 5 | 8.0*** | 13.4*** | 16.6*** |

*P from 0.05 to 0.01;
**P from 0.01 to 0.001;
***P < 0.001.
D₉₅ is the root depth measures the depth where 95% of root length in mesocosms,
Irrigation is the moisture regimes imposed;
Gen is the genotype;
RWC is the leaf relative water content (%)

CCS Effect on Plant Growth and Yield

In the greenhouse, overall plant performance was assessed using shoot biomass. The low water availability in water-stressed conditions resulted in shoot biomass reductions (relative to well-watered) of 42% in EXPII and 46% in EXPIII (Table 1 and FIG. 9). Under water stress lines with large CCS had 34% (EXPII) and 44% (EXPIII) greater shoot biomass than lines with small CCS (Table 1 and FIG. 9).

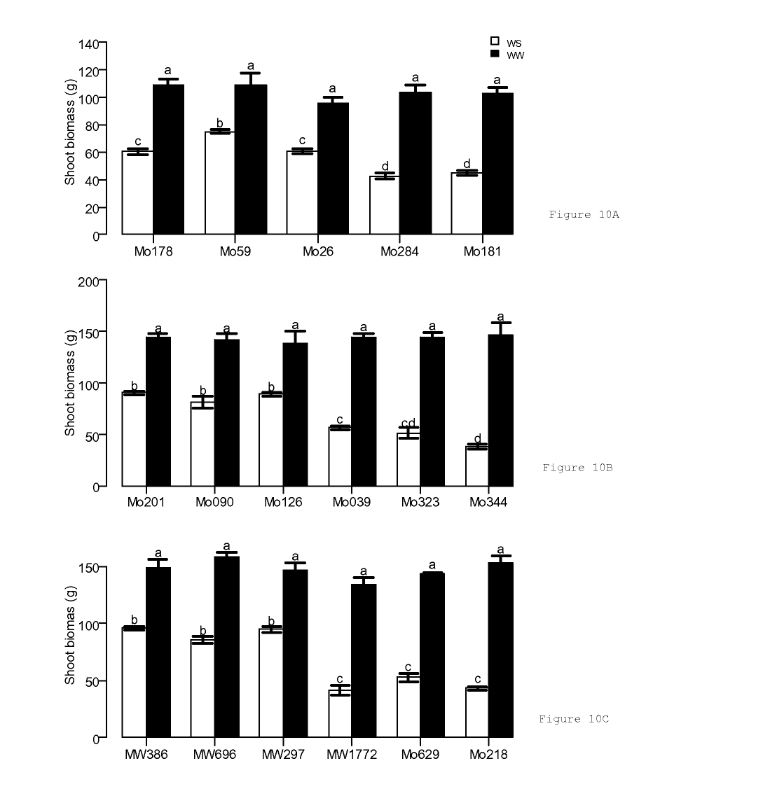

In the field, low water availability in water-stressed conditions resulted in a shoot biomass reductions of 46% (PA1), 38% (PA2) and 53% (MW2-2), 70 days after planting (Table 2 and FIG. 10A-10C). Under water stress, lines with large CCS had greater shoot biomass than lines with small CCS by 33% (PA1), 36% (PA2) and 100% (MW2-2). However, there were no significant differences in well watered conditions (FIG. 10).

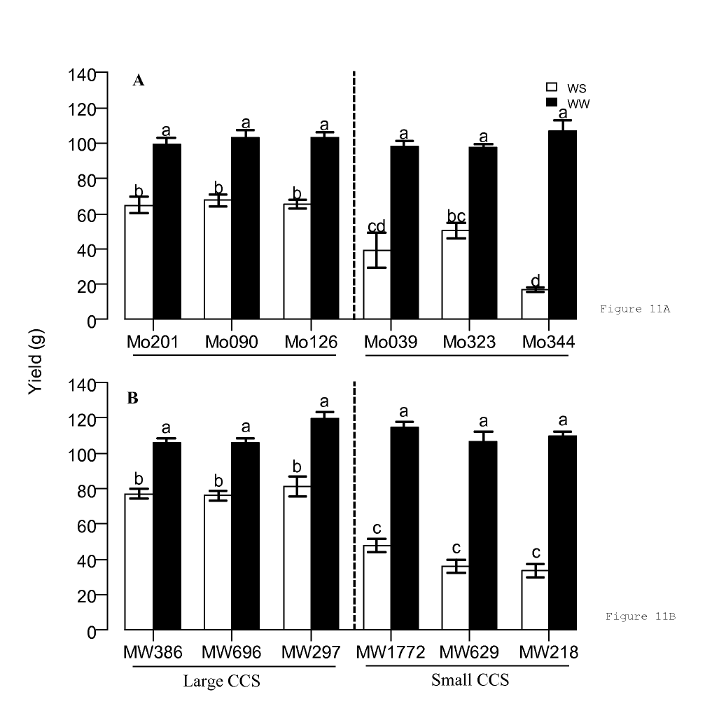

Water stress significantly reduced grain yield in both trials with the reduction in yield ranging from 32% to 82% in the first year and from 26% to 69% in the second year. In both trials, large variation was observed in mean grain yield under drought stress (FIGS. 11A and 11B). Under water stress, lines with large CCS had greater yield compared to lines with small CCS by 82% (PA2) and 99% (MW2-2) (FIG. 11).

### Example 3

### Materials and Methods for Assessing Root Cortical Cell File Number Under Drought

Plant Materials

The cortex of the maize root is composed of several concentric layers of parenchyma cells, the number of which is referred to as ‘cortical cell file number’ (CCFN) herein. Laser ablation tomography and semi-automated image analysis in RootScan was used to select populations of maize plants with contrasting CCFN. Based on preliminary experiments conducted under optimal conditions in the field and greenhouse, a set of six IBM lines contrasting in CCFN was selected for experiments for one year and another set of six IBM lines also contrasting in CCFN was selected for experiments in the consecutive year (Table 4). In Malawi, the experimental material consisted of a set of 33 lines (Table 4). These lines were a subset of a larger group of the Malawi maize breeding program and selected to represent a broad set of gene pools and diversity contrasting in CCFN.

TABLE 4
Lines Used in Experiments

| Experiment | Lines/entries |
|---|---|
| Experiment I | NyH128, NyH39, NyH126, NyH246, NyH158, |
| | NyH195, NyH237, NyH35 NyH54, NyH220, NyH225 |
| | Mo21, Mo344, Mo205, Mo352, Mo323, Mo345, |
| | Mo178, Mo358, Mo201, Mo121, Mo150, Mo48, |
| | Mo86, Mo263, Mo98 |
| Experiment II | Mo129, Mo132, Mo181, Mo233, Mo317, Mo365 |
| & Field 1 | |
| Experiment | Mo048, Mo178, M0277, Mo263, Mo146, Mo345 |
| III & Field 2 | |
| Field - | AR403-3, AR660, CML196, CML247, CML321, |
| Malawi | CML339, CML344, CML373,, CML442, CML511, |
| | CZL99011, M70-5-2, M70-6-2, M70-9-1, MANICA-4, |
| | MAT273-4-2-1, ZM523, 46C2W, AR239-2, AR267, |
| | AR424(5012), AR716, AR858, CML199, CML377, |
| | E21, M70-29-2, M70-29-3, M73-18, Mkangala, |
| | SC513, SW 19 |

Greenhouse Experiment

Three experiments were conducted under the same conditions in two consecutive years as described in Example 1. Root images obtained as described in Example 1 were analyzed using RootScan, an image analysis tool developed for analyzing root anatomy (Burton et al., Plant and Soil, 357: 189-203, 2012). The CCFN was determined from three different images per root segment. CCFN was obtained by counting the cell layers from the epidermis to the endodermis.

### Experiment I

The aim of this experiment was to assess the relationship between phenotypic variation for CCFN and root respiration. A randomized complete block design with time of planting as a blocking factor replicated three times was used. A set of 25 genotypes (Table 4) contrasting in CCFN was planted under mild water stress conditions. Water stress was imposed by withholding water 14 days after planting. Plants were harvested for root respiration measurements 35 days after planting.

### Experiment II and III

Two experiments were conducted, one in the fall (experiment II) and the following summer (experiment III). A set of six genotypes was planted in each experiment (Table 4). A randomized complete block design, with time of planting as a blocking factor with four replications was used in both experiments. Planting was staggered by seven days. As with experiments II and III in Example 1, the irrigated mesocosms (control) each received 200 ml of water every other day, to replenish water lost by evapotranspiration, and in stressed mesocosms, water application was withheld five days after planting to allow the plants to exploit residual moisture to simulate terminal drought. Stomatal conductance was measured as described in Example 1. Plants were harvested 30 days after planting for root respiration measurements, root growth distribution and shoot biomass. The dry matter of the shoot and root were measured after drying at 70° C. for 72 h and root length distribution was determined as described above.

Field Experiments-Rock Springs, Pa., USA

The field experiments were conducted in rainout shelters located at the Russell E. Larson Agricultural Research Center in Rock Springs, Pa., USA (40° 42′37″0.52 N, 77° 57′07″0.54 W) as described in Example 1. Soil water content for both well watered and water stressed treatments was monitored regularly during the experiment. In the first field experiment, soil water content was monitored using Time Domain Reflectometery (TDR) probes installed at 20 and 40 cm soil depth while in the second field experiment, soil water content was monitored using the TRIME FM system (IMKO Micromodultechnik GmbH, Ettlingen, Germany) at three depths (20, 35 and 50 cm) both inside and outside the rainout shelter. Seven readings were taken between 30 to 120 days after planting. In both years the experiments were arranged as a randomized complete block split plot design with four replications. The main plots were composed of two moisture regimes and the subplots contained six genotypes contrasting in CCFN in each experiment. Each subplot consisted of three rows, with each row being 2.5 m long, with a row spacing of 0.75 m. The drought treatment was initiated 35-40 days after planting using an automated rainout shelter. The shelters (10 by 30 m) were covered with a clear greenhouse plastic film (0.184 mm) and were automatically triggered by rainfall to cover the plots, excluding natural precipitation. Adjacent non-sheltered control plots were rainfed and drip-irrigated when necessary to maintain the soil moisture close to field capacity throughout the growing season.

Plant Measurements

Leaf RWC, soil cores, and shoot and roots were evaluated as described in Example 1. Three 8-cm root segments were collected 10-20 cm from the base of a representative second whorl crown root of each plant, and used to assess cortical cell file number. The segments were preserved in 75% alcohol before being processed as described above. At physiological maturity grain yield was collected each plot.

Field Experiments—Malawi

Evaluation of Phenotypic Variation of Cortical Cell File in Malawi Germplasm

A set of 151 maize genotypes obtained from the Department of Agricultural Research and Technical Services, Malawi was planted at Bunda (14° 10′26.76″S, 33° 48′01.85″) in two consecutive years under optimum conditions. Briefly, these lines were assembled from 30 CIMMYT lines, 40 lines from the Malawi maize breeding program, and 81 landraces collected across Malawi. In both years, the experiments were arranged as randomized complete design with three replications. Each plot consisted of a single 6 m long row with 25 plants. Roots were sampled 70 days after planting. Three representative plants of each plot were excavated and evaluated as described in Example 1. Root segments were collected from 10-20 cm from the base of three representative second whorl crown roots per plant for CCFN determination. The samples were preserved in 75% alcohol and processed as described above.

Utility of Cortical Cell File Number Under Water Limited Conditions in Two Agroecological Zones in Malawi

Two sites were selected in central Malawi: Chitala (13° 28′49.82″S, 33° 59′47.66″E,) and Bunda (14° 10′26.76″S, 33° 48′01.85″), representing two agroecological zones for maize cultivation. Soils at both sites were classified as oxic rhodustalfs. The experiments were conducted during the summer (i.e. rain-free period August to November). A set of 33 maize genotypes contrasting in CCFN was planted at each site. The experiments were arranged as split-plots in a randomized complete block design with four replications. The main plots were composed of two moisture regimes and the subplots contained 33 genotypes contrasting in CCFN. Seeds were planted, watered, and fertilized as described in Example 1. Shoot biomass and leaf relative water content of the third fully expanded leaf was determined 70 days after planting at both sites. Leaf relative water content was determined 60 days after planting as described above. Root segments for anatomical analysis were collected 70 days after planting and shipped to Penn State University where they were processed as described above. At physiological maturity grain yield was collected each plot.

Data Analysis

The data from each year were analyzed separately considering that different sets of genotypes were used as described in Example 1. Linear regression analysis was used to establish relationships between CCFN and measured and calculated parameters.

### Example 4

### Phenotypic Variation for CCFN in Maize

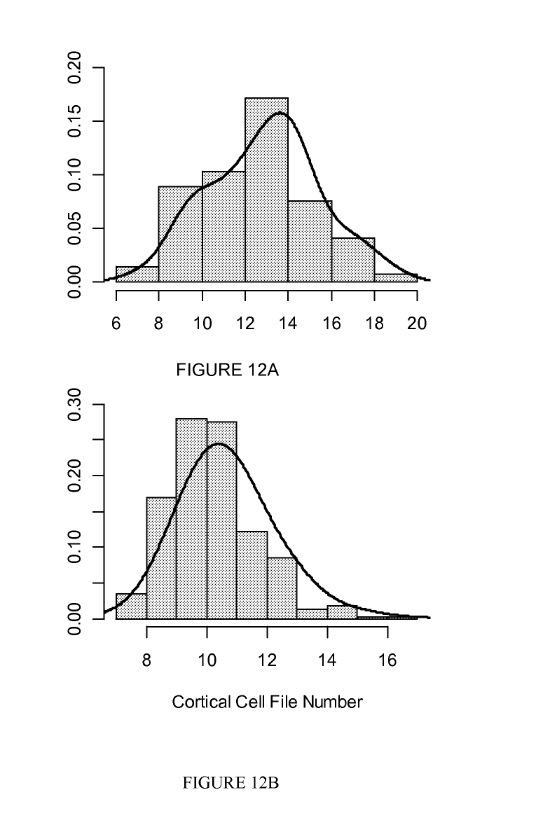

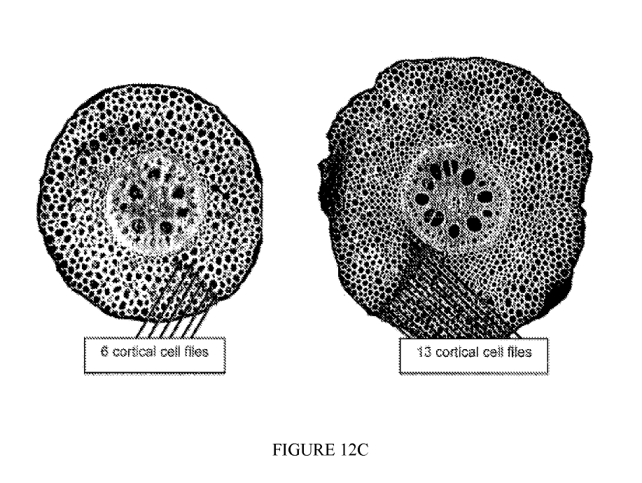

There was substantial phenotypic variation for CCFN within maize landraces and recombinant inbred lines (RILs) (FIG. 12). Among landraces, the variation was over 3-fold while for RILs, variation was over 2-fold. The CCFN ranged from 6 to 19 in landraces (FIG. 12A) and for RILs, CCFN ranged from 8 to 17 (FIG. 12B). The frequency distributions of the CCFN showed continuous variation with approximately normal distributions (FIG. 12). FIG. 12C contains cross sections of maize roots showing genotypic difference in CCFN (6 cortical cell files vs. 13 cortical cell files) using images obtained from laser ablation tomography. Sections are from maize crown roots grown in the field 70 days after planting.

The trait was consistent across environments and sites. The correlation coefficients (r) were calculated between CCFN determined from 70 and 30 day old plants (i.e. field and greenhouse respectively) and across sites in Malawi. A positive correlation between CCFN for greenhouse and field plants was observed (R²=0.85, P<0.05), likewise CCFN across two field sites in Malawi were significantly correlated (R²=0.68, P<0.05), suggesting stability of the trait in our environments.

Greenhouse Study

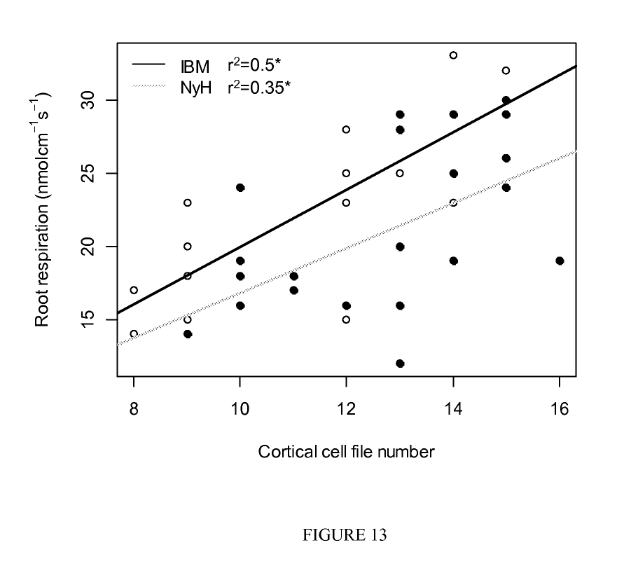

Root respiration was measured in experiment II and III, the pattern of results obtained were similar as the results of experiment I, hence only the results of experiment I are reported here for brevity. Furthermore, in experiment I more genotypes were used. Root respiration rates varied widely, ranging from 13 to 32 nmol CO₂ cm⁻¹ s⁻¹ for IBM lines, and from 12 to 29 nmol CO₂ cm⁻¹ s⁻¹ for NyH lines (FIG. 13). Respiration rates decreased substantially with decreasing CCFN (FIG. 13). For examples roots with 8 cell files respired 57% less than roots with 16 cell files.

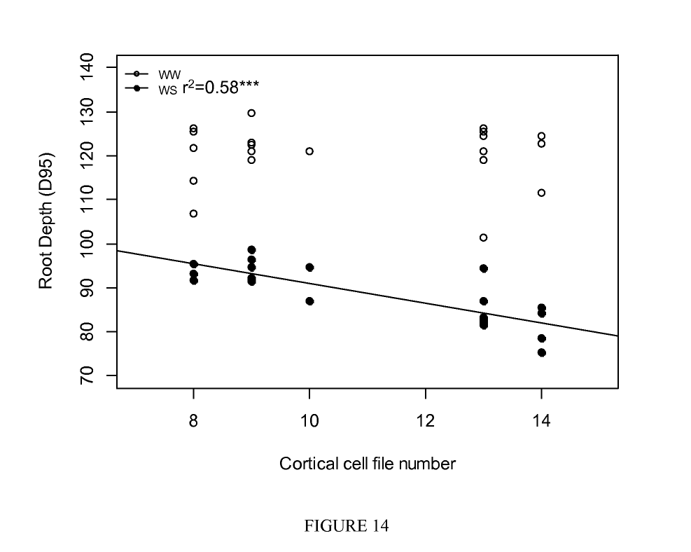

In well watered conditions, roots reached the bottom of the mesocosms 30 days after planting. In contrast, in water stressed conditions very few genotypes reached a depth of 90 cm. Linear regressions were used to estimate the effect of CCFN on rooting depth expressed as D₉₅. Rooting depth decreased linearly with CCFN in water stressed conditions (FIG. 14), but there was no relationship in well watered conditions (FIG. 14).

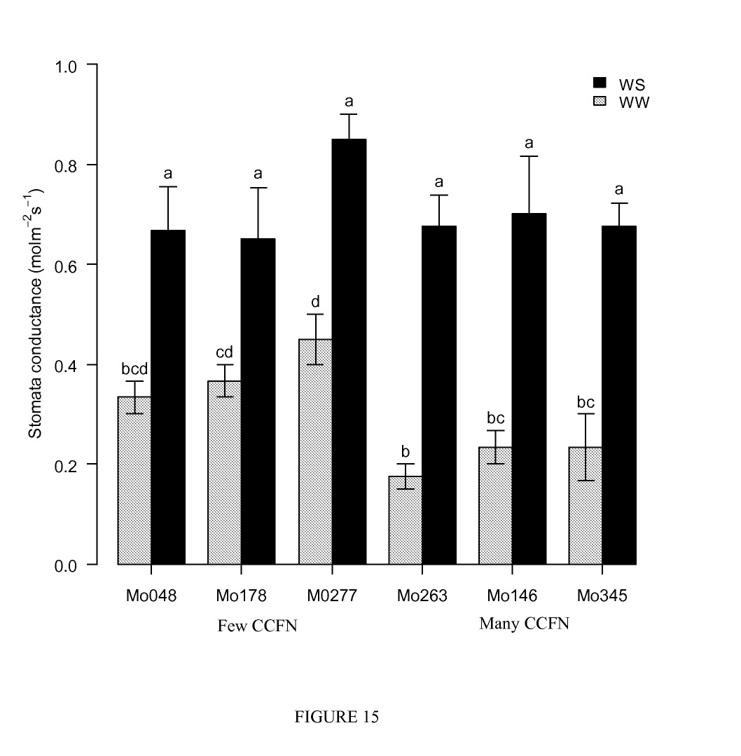

The low water availability in water stressed conditions resulted in 58% reduction of stomatal conductance (FIG. 15). Under water stress conditions lines with few cell files had 78% greater stomatal conductance than lines with many cell files, while there was no difference under well watered conditions (FIG. 15).

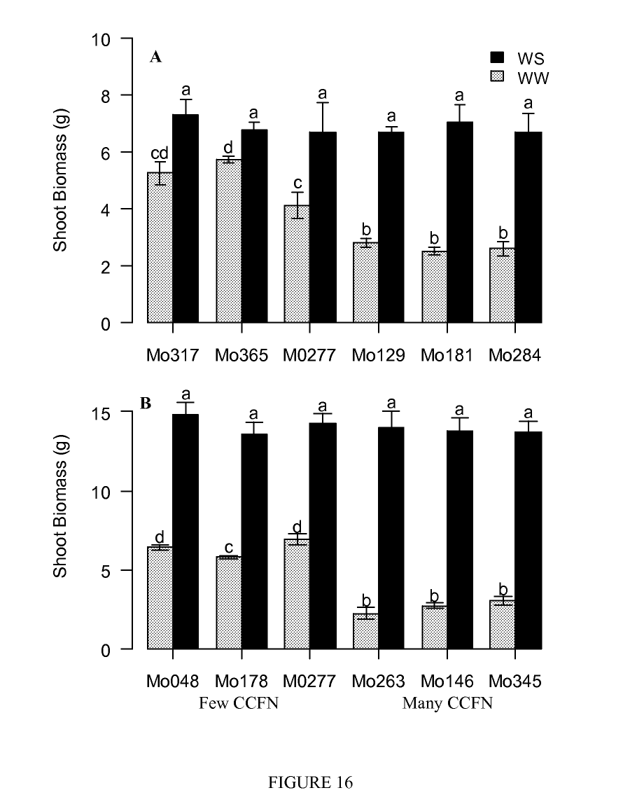

Water stress reduced shoot biomass in all genotypes (FIG. 16) in both experiments. Under water stress, lines with few cell files were superior in shoot biomass production than genotypes with many cortical cell files (FIG. 16). Reduced cell file lines had 52% and 139% greater biomass than lines with many cell files in experiment II and III respectively (FIG. 15). CCFN had no effect on biomass under well watered conditions.

Field Experiments—Rock Springs Pa.

Soil moisture was maintained between 0.234 cm³ cm⁻³ and 0.227 cm³ cm⁻³ at 0-15 cm and 0.352 cm³ cm⁻³ and 0.350 cm³ cm⁻³ at 30-50 cm under well-watered conditions (Table 5). A gradual decrease from 0.231 cm³ cm⁻³ to 0.151 cm³ cm⁻³ at 0-15 cm and 0.348 cm³ cm⁻³ to 0.250 cm³ cm⁻³ at 30-50 cm was observed in water stressed plots. A clear distinction between soil moisture levels from well watered and stressed plots was noted at 40, 50, 60, 70, 80 and 90 days after planting.

TABLE 5
Soil moisture content in different layers of
the soil profile-field 2 experiment in PA.

| | |
|---|---|
| Moisture at | Soil Moisture (cm³cm⁻³) |

| DAP¹ | Treatment | 0-15 | 15-30 | 30-50 |
|---|---|---|---|---|
| 30 | WW | 0.234 | 0.302 | 0.352 |
| | WS | 0.231 | 0.300 | 0.348 |
| 40 | WW | 0.230 | 0.303 | 0.355 |
| | WS | 0.200 | 0.255 | 0.346 |
| 50 | WW | 0.237 | 0.308 | 0.356 |
| | WS | 0.195 | 0.222 | 0.336 |
| 60 | WW | 0.230 | 0.306 | 0.356 |
| | WS | 0.170 | 0.208 | 0.307 |
| 70 | WW | 0.226 | 0.301 | 0.354 |
| | WS | 0.151 | 0.201 | 0.280 |
| 80 | WW | 0.229 | 0.302 | 0.352 |
| | WS | 0.120 | 0.200 | 0.275 |
| 90 | WW | 0.227 | 0.300 | 0.350 |
| | WS | 0.112 | 0.200 | 0.250 |

¹DAP = days after planting

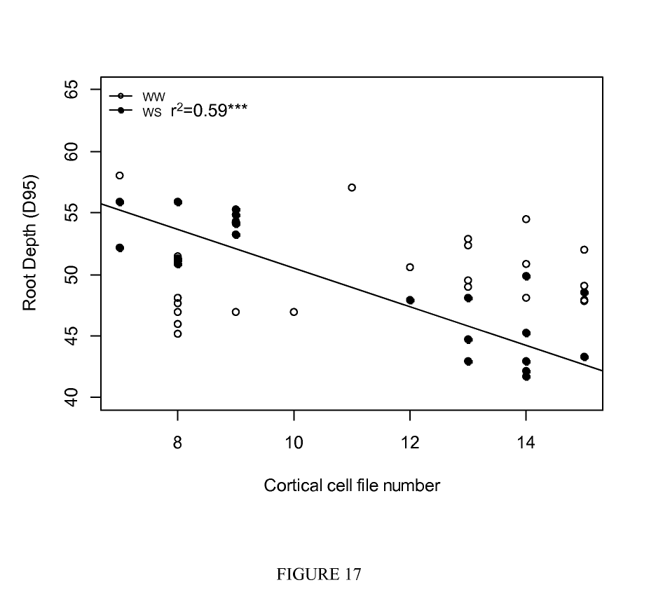

CCFN had no effect on root depth under well watered conditions, but under water stress greater CCFN reduced root depth expressed as D₉₅ (FIG. 17). Lines with 7 cell files had 33% deeper D₉₅ than lines with 16 cell files (FIG. 17).

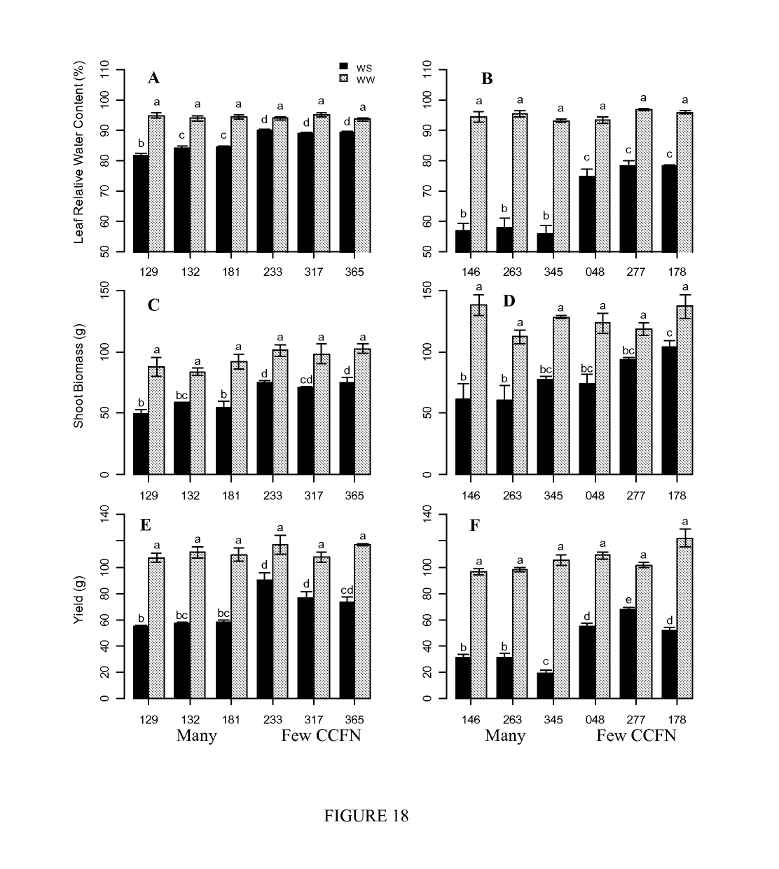

Leaf RWC of well-watered plants averaged about 90% at 60 days after planting with no significant differences among genotypes (FIG. 18 A,B). Water stress significantly reduced RWC for all genotypes relative to the well-watered plants, and significant differences among genotypes were observed (FIG. 18 A,B). Under water stress, lines with few cell files had better plant water status than those having many cell files (FIG. 18 A,B). In addition, there was a significant and positive correlation between D₉₅ and leaf RWC in water stress conditions (r²=0.51, p<0.000), lines with deeper D₉₅ having better leaf water status than lines with shallow D₉₅, while there was no relationship in well watered conditions.

Water stress reduced shoot biomass by 30% in the field 1 experiment and 33% in the field 2 experiment. Analysis of variance indicated that significant differences existed among genotypes under water stress and that there was no significant differences between genotypes in well watered conditions (FIG. 18 C, D). Reduced CCFN lines had 35% and 45% greater shoot biomass than lines with many cell files in the field 1 and 2 experiments, respectively under water stress (FIG. 18 C,D).

Water stress significantly reduced grain yield in both trials with the reduction in yield ranging from 26% to 68% in the field 1 experiment and from 33% to 75% in the field 2 experiment compared with well-watered plants. In both trials large variation was observed in mean grain yield under drought stress (FIG. 18 E, F). Reduced CCFN lines had 38% and 114% greater yield than lines with many cell files in water stressed conditions in field 1 and 2 experiments, respectively.

Utility of CCFN Under Different Agroecologies in Malawi

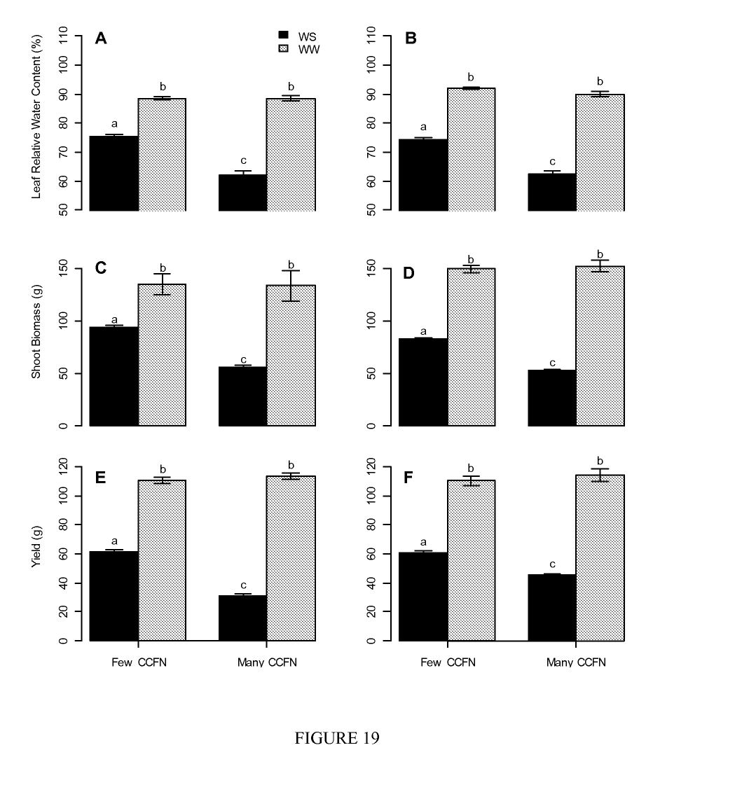

A set of 33 maize lines was grown in two agroecological zones representative of the maize growing environments in Malawi. These genotypes were a subset of a larger group of the Malawi maize breeding program and selected to represent a broad set of genetic diversity and contrasting CCFN. Significant effects of irrigation level, genotype and their interactions were observed for yield, shoot biomass, and leaf relative water content (FIG. 19).

Under water stress lines with reduced CCFN had 20% and 19% greater leaf relative water content than lines with many cell files in Bunda and Chitala respectively (FIG. 19 A, B). Water stress resulted in 43% and 54% reduction in shoot biomass in Bunda and Chitala respectively. Reduced CCFN lines had 70% and 57% greater shoot biomass than lines with many cell files (FIG. 19 C, D). Yield was reduced by 59% in Bunda and by 53% in Chitala by water stress. Reduced CCFN lines had 93% and 33% greater yield than lines with many cell files under water stress (FIG. 19 E, F).

### Example 5

### Genome-Wide Association Study

Data was collected for root anatomical traits, including CCFN for four consecutive years of samples (i.e. 12,000 plots, 36,000 individual plants phenotyped) by laser ablation tomography and semi-automated image analysis in RootScan. Crop agronomy, phenotyping system, and image analysis improved in the final 3 years over the first year, resulting in higher repeatability and better quality of data (e.g. repeatability of CCFN among replicates in the final year was double that in the first year). Heritability of CCFN across the 4 years was 0.37. Genotypes were significantly different in CCFN (p<0.01).

A genome-wide associating study (GWAS) was performed with 438 k SNP markers derived from RNA sequence. Candidate genes were prioritized based on 1) expression profiles using an expanded gene atlas including diverse root tissues, 2) functional annotation in maize and in rice orthologs, and 3) analysis of orthologous function in Arabidopsis. Candidate genes for CCFN were identified on chromosome 5, 6, and 8. Significant SNPs were associated with Maize Gene models: GRMZM2Gl19133; GRMZM2G125976; GRMZM2G077498; GRMZM2Gl06250; GRMZM2G067830 (protein degradation, ubiquitin E3 ring); GRMZM2G011169 (development); GRMZM2G070199; GRMZM2G031952; GRMZM2G0118037 (protein degradation, subtilases); GRMZM2G063961; and GRMZM2G302778.

Interestingly one of the SNPs on chromosome 6 (gene model:GRMZM2G070199) was located close to SCARECROW (SCR), which has been shown to be involved in root apical meristem and radial development in maize and Arabidopsis (Lim et al., 2000, The Plant cell, 12, 1307-1318). Moreover, GRMZM2G070199 is highly expressed in the primary roots of maize seedlings

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method for producing a drought tolerant plant, said method comprising a) selecting a plant having (i) a reduced root cortical cell file number (CCFN) from a plurality of plants or (ii) a larger average root cortical cell size (CCS) area from a plurality of plants; and b) producing a progeny of said selected plant.

2. The method of claim 1, wherein said progeny is a seed, wherein upon planting said seed, the resulting plant is drought tolerant.

3. The method of claim 1, wherein said progeny is produced by cross-pollinating the selected plant with a different plant of the same species.

4. The method of claim 1, wherein said progeny is produced by self-pollinating the selected plant.

5. The method of claim 1, wherein said plant is a monocot.

6. The method of claim 5, wherein said monocot is selected from the group consisting of an Agrostis sps. (bent grass), an Andropogon sps. (blue stem grass), an Arundo sps. (cane), an Avena sps. (oats), a Cynodon sps. (Bermuda grass), an Elaeis sps. (oil palm), an Eragrostis sps. (love grass), a Festuca sps. (fescue), a Hordeum sps., a Lolium sps. (rye grass), a Miscanthus sps., an Oryza sps., a Panicum sps., a Pennisetum sps. (fountain grass), a Poa sps., a Saccharum sps., a Secale sps., a Sorghum sps., a Triticum sps. (wheat), a Zea sps., and a Zoysia sps.

7. The method of claim 1, wherein said plant is Hordeum vulgare, Oryza sativa, Panicum miliaceum, Panicum virgatum, Saccharum officinarum, Secale cereal, Sorghum bicolor, Triticum aestivum, Triticum durum, Triticum spelta, or Zea mays.

8. The method of claim 1, wherein said plant is a Zea mays plant.

9. The method of claim 8, wherein said CCFN of said selected plant is 10 or less.

10. The method of claim 9, wherein said CCFN of said selected plant is 9 or less.

11. The method of claim 9, wherein said CCFN of said selected plant is 6-8.

12. The method of claim 8, wherein said CCS area of said selected plant is greater than 230 μm².

13. The method of claim 12, wherein said CCS area of said selected plant is greater than 300 μm².

14. The method of claim 12, wherein said CCS area of said selected plant is greater than 350 μm².

15. The method of claim 12, wherein said CCS area of said selected plant is greater than 400 μm².

16. The method of claim 1, wherein said plant is a dicot.

17. The method of claim 16, wherein said dicot is selected from the group consisting of a Phaseolus sps., a Vigna sps., a Gossypium sps., a Medicago sps., a Helianthus sps., a Brassica sps., a Glycine sps., or a Carthamus sps.

18. The method of claim 17, wherein said plant is a Phaseolus vulgaris, Vigna radiata, Medicago sativa, Helianthus annuus, Brassica rapa, Brassica napus, Glycine max, or Carthamus tinctorius.

19. A method of producing a maize plant, said method comprising
a) obtaining one or more first maize parent plants having (i) a root cortical cell file number (CCFN) of 10 or less or (ii) an average root cortical cell size (CCS) area greater than 230 μm2;
b) obtaining one or more second maize parent plants; and
c) crossing the one or more first parent plants and the one or more second parent plants to produce progeny, wherein said progeny have drought tolerance.

20-24. (canceled)

25. The method of claim 19, wherein said first and second parent plants are inbred lines.

26-27. (canceled)

28. A method of producing a maize plant, said method comprising
a) obtaining one or more first maize parent plants having (i) a root cortical cell file number (CCFN) of 10 or less or (ii) an average root cortical cell size (CCS) area greater than 230 μm²;
b) obtaining one or more second maize parent plants;
c) crossing the one or more first parent plants and the one or more second parent plants; and
d) selecting, for one to five generations, for progeny plants having drought tolerance.
